(19) United States
(12) Patent Application Publication
(10) Pub. No.: US 2011/0190313 A1
(43) Pub. Date: Aug. 4, 2011

(54) Treatment of Pulmonary Arterial Hypertension

(76) Inventors: Steve Pascoe, Alsace (FR); Deborah Quinn, Nahant, MA (US)

(21) Appl. No.: 13/058,742

(22) PCT Filed: Aug. 11, 2009
(86) PCT No.: PCT/US2009/053358

§ 371 Date: Mar. 31, 2011

Related U.S. Application Data
(60) Provisional application No. 61/088,382, filed on Aug. 13, 2008.
(60) Provisional application No. 61/164,501, filed on Mar. 30, 2009.

(51) Int. Cl. C07D 401/14; A61K 31/496; A61P 9/12

(52) U.S. Cl. 514/252.18; 544/357

(57) ABSTRACT

The present invention pertains to the use of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof or a pyrimidylaminobenzamide of formula I

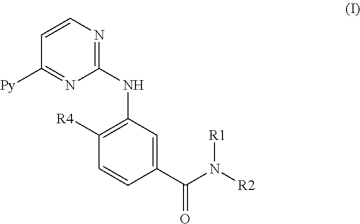

wherein the radicals and symbols are as defined herein, or a pharmaceutically acceptable salt thereof, for the manufacture of a medicament for treating pulmonary arterial hypertension (PAH), especially in patients who failed prior PAH therapy.

The invention relates to the use of 4-(4-methylpiperazin-1-ylmethyl)-N-[4-methyl-3-(4-pyridin-3-yl)pyrimidin-2-ylamino)phenyl]-benzamide (also known as “Imatinib” [International Non-proprietary Name]; hereinafter: “COMPOUND I”) or a pharmaceutically acceptable salt thereof or a pyrimidylaminobenzamide of formula I as defined below or a pharmaceutically acceptable salt thereof for the manufacture of a medicament for the treatment of pulmonary arterial hypertension, to COMPOUND I or a pharmaceutically acceptable salt thereof or a pyrimidylaminobenzamide of formula I as defined below or a pharmaceutically acceptable salt thereof for the treatment of pulmonary arterial hypertension, and to a method of treating warm-blooded animals including humans suffering from pulmonary arterial hypertension, by administering to a said animal in need of such treatment an effective dose of COMPOUND I or a pyrimidylaminobenzamide of formula I or a pharmaceutically acceptable salt thereof.

Pulmonary arterial hypertension is a life-threatening disease characterized by a marked and sustained elevation of pulmonary artery pressure. The disease results in right ventricular (RV) failure and death. Current therapeutic approaches for the treatment of chronic pulmonary arterial hypertension mainly provide symptomatic relief, as well as some improvement of prognosis. Although postulated for all treatments, evidence for direct anti-proliferative effects of most approaches is missing. In addition, the use of most of the currently applied agents is hampered by either undesired side effects or inconvenient drug administration routes. Pathological changes of hypertensive pulmonary arteries include endothelial injury, proliferation and hyper-contraction of vascular smooth muscle cells (SMCs).

The instant invention is a response to the need for an alternative therapy in the treatment of pulmonary hypertension, especially pulmonary arterial hypertension.

United States patent specification US 2006/0154936 disclosed the use of COMPOUND I alone or in combination with other medication as an alternative to existing therapies for the treatment of pulmonary hypertension.

It has now surprisingly been demonstrated that pulmonary arterial hypertension can be successfully treated with COMPOUND I, or pharmaceutically acceptable salt thereof or a pyrimidylaminobenzamide of formula I or a pharmaceutically acceptable salt thereof, in particular in patients who failed prior therapy.

In a first aspect the present invention concerns the use of COMPOUND I having the formula

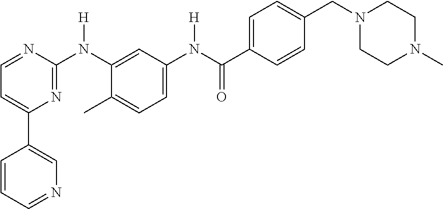

or a pharmaceutically acceptable salt thereof, or a pyrimidylaminobenzamide of formula I

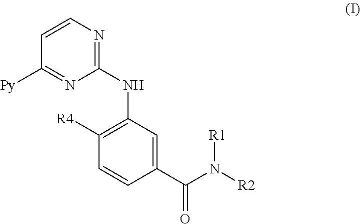

wherein
Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
or a pharmaceutically acceptable salt thereof, for the manufacture of a medicament for treating pulmonary arterial hypertension, especially in patients who failed prior PAH therapy.

In a second aspect the present invention concerns 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof, or a pyrimidylaminobenzamide of formula I as defined above or a pharmaceutically acceptable salt thereof, for use in treating pulmonary arterial hypertension (PAH) in patients who failed prior PAH therapy.

In a third aspect the present invention concerns a method of treating warm-blooded animals including humans suffering from pulmonary arterial hypertension, by administering to a said animal in need of such treatment an effective dose of 4-(4-methylpiperazin-1-ylmethyl)-N-[4-methyl-3-(4-pyridin-3-yl)pyrimidin-2-ylamino)phenyl]benzamide or a pharmaceutically acceptable salt thereof or a pyrimidylamino-benzamide of formula I as defined above or a pharmaceutically acceptable salt thereof.

In a fourth aspect the present invention concerns a method of treating a human suffering from
(a) idiopathic or primary pulmonary hypertension,
(b) familial hypertension,
(c) pulmonary hypertension secondary to, but not limited to, connective tissue disease, congenital heart defects (shunts), pulmonary fibrosis, portal hypertension, HIV infection, sickle cell disease, drugs and toxins (e.g., anorexigens, cocaine), chronic hypoxia, chronic pulmonary obstructive disease, sleep apnea, and schistosomiasis,
(d) pulmonary hypertension associated with significant venous or capillary involvement (pulmonary veno-occlusive disease, pulmonary capillary hemangiomatosis),
(e) secondary pulmonary hypertension that is out of proportion to the degree of left ventricular dysfunction,
(f) persistent pulmonary hypertension in newborn babies,
   especially in patients who failed prior PAH therapy, which comprises administering to said human in need of such treatment a dose effective against the respective disorder of 4-methylpiperazin-1-ylmethyl)-N-[4-methyl-3-(4-pyridin-3-yl)pyrimidin-2-ylamino)phenyl]-benzamide or a pyrimidylaminobenzamide of formula I as defined above or a pharmaceutically acceptable salt thereof.

The preparation of COMPOUND I and the use thereof, especially as an anti-tumor agent, are described in Example 21 of European patent application EP-A-0 564 409, the contents of which is hereby incorporated by reference, and in corresponding applications and patents in numerous other countries, e.g. in U.S. Pat. No. 5,521,184 and in Japanese patent 2706682.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

The monomethanesulfonic acid addition salt of COMPOUND I (hereinafter “COMPOUND I mesylate” or “imatinib mesylate” or “COMPOUND I monomethanesulfonate”) and a preferred crystal form thereof, e.g. the β-crystal form, are described in PCT patent application WO99/03854 published on Jan. 28, 1999.

Possible pharmaceutical preparations, containing an effective amount of COMPOUND I or a pharmaceutically acceptable salt thereof are also described in WO99/03854, the contents of which is incorporated herein by reference.

According to formula I, the following suitable, preferred, more preferred or most preferred aspects of the invention may be incorporated independently, collectively or in any combination.

Preference is also given to pyrimidylaminobenzamides of formula I, wherein py is 3-pyridyl and wherein the radicals mutually independently of each other have the following meanings:
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl; more preferably hydrogen;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
R₄ represents lower alkyl, especially methyl.

A preferred pyrimidylaminobenzamide of formula I is 4-methyl-3-[[4-(3-pyridinyl)-2-pyrimidinyl]amino]-N-[5-(4-methyl-1H-imidazol-1-yl)-3-(trifluoromethyl)phenyl]benzamide, also known as “nilotinib”.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g. by methyl, by halogen-lower alkyl, e.g. trifluoromethyl, or by phenyl; lower alkylene dioxy bound to two adjacent C-atoms, e.g. methylenedioxy, lower alkyl, e.g. methyl or propyl; halogen-lower alkyl, e.g. trifluoromethyl; hydroxy-lower alkyl, e.g. hydroxymethyl or 2-hydroxy-2-propyl; lower alkoxy-lower alkyl; e.g. methoxymethyl or 2-methoxyethyl; lower alkoxycarbonyl-lower alkyl, e.g. methoxy-carbonylmethyl; lower alkynyl, such as 1-propynyl; esterified carboxy, especially lower alkoxycarbonyl, e.g. methoxycarbonyl, n-propoxy carbonyl or iso-propoxy carbonyl; N-mono-substituted carbamoyl, in particular carbamoyl monosubstituted by lower alkyl, e.g. methyl, n-propyl or iso-propyl; amino; lower alkylamino, e.g. methylamino; di-lower alkylamino, e.g. dimethylamino or diethylamino; lower alkylene-amino, e.g. pyrrolidino or piperidino; lower oxaalkylene-amino, e.g. morpholino, lower azaalkylene-amino, e.g. piperazino, acylamino, e.g. acetylamino or benzoylamino; lower alkylsulfonyl, e.g. methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen. N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen. N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino. N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino. N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g. pyrrolidino, 2-oxopyrrolidino or piperidino; lower oxaalkylene-amino, e.g. morpholino, or lower azaalkylene-amino, e.g. piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic hetero-aryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula I and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substituents selected from the group defined above as substituents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)-2-one. In another preferred embodiment, the pyrimidinyl radical is substituted by hydroxy both in position 2 and 4 and hence exists in several tautomeric forms, e.g. as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1H-azepin-3-yl, 2-tetrahydrofuranyl, or 2-methyl-1,3-dioxolan-2-yl.

Pyrimidylaminobenzamides within the scope of formula I, wherein Py is 3-pyridyl and the process for their manufacture are disclosed in WO 04/005281, the contents of which is incorporated herein by reference.

Pharmaceutically acceptable salts of pyrimidylaminobenzamides of formula I, wherein Py is 3-pyridyl, are especially those disclosed in WO2007/015871. In one preferred embodiment nilotinib is employed in the form of its hydrochloride monohydrate. WO2007/015870 discloses certain polymorphs of nilotinib and pharmaceutically acceptable salts thereof useful for the present invention.

The pyrimidylaminobenzamides of formula I, wherein Py is 3-pyridyl, can be administered by any route including orally, parenterally, e.g., intraperitoneally, intravenously, intramuscularly, subcutaneously, intratumorally, or rectally, or enterally. Preferably, the pyrimidyl-aminobenzamides of formula I, wherein py is 3-pyridyl, is administered orally, preferably at a daily dosage of 50-2000 mg. A preferred oral daily dosage of nilotinib is 200-1200 mg, e.g. 800 mg, administered as a single dose or divided into multiple doses, such as twice daily dosing.

The term “treatment” as used herein means curative treatment and prophylactic treatment.

The term “curative” as used herein means efficacy in treating ongoing episodes of pulmonary hypertension, especially pulmonary arterial hypertension.

The term “prophylactic” means the prevention of the onset or recurrence of pulmonary hypertension, especially pulmonary arterial hypertension.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The invention also pertains to a pharmaceutical preparation for the treatment of pulmonary arterial hypertension comprising COMPOUND I.

### SHORT DESCRIPTION OF THE FIGURES

FIG. 1 depicts the change in pulmonary vascular resistance (PVR) in patients obtaining Imatinib mesylate.

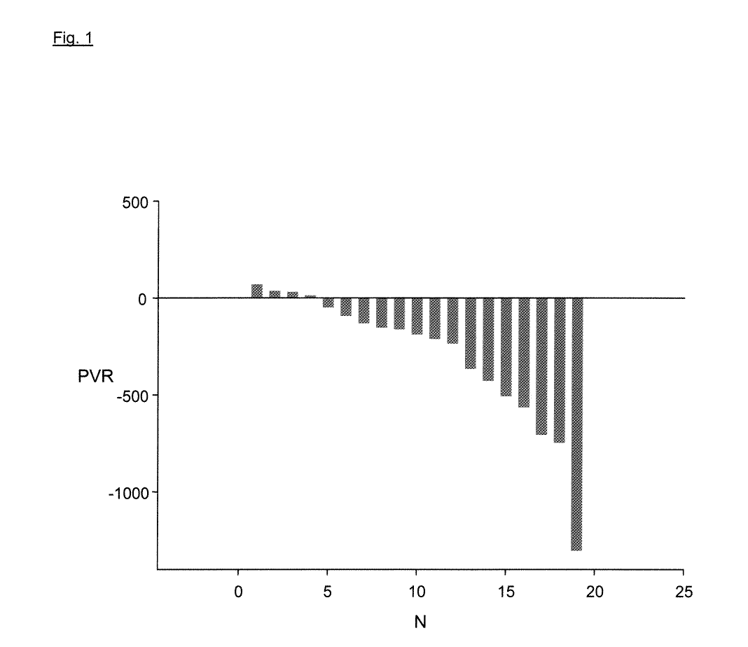

FIG. 2 depicts the change in pulmonary vascular resistance (PVR) in patients obtaining placebo.

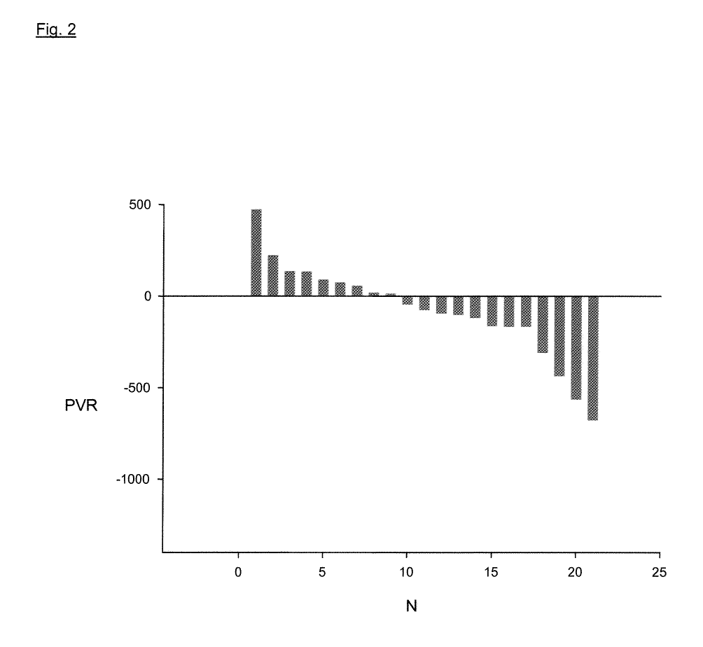

FIG. 3 depicts the change in cardiac output (CO) in patients obtaining Imatinib mesylate.

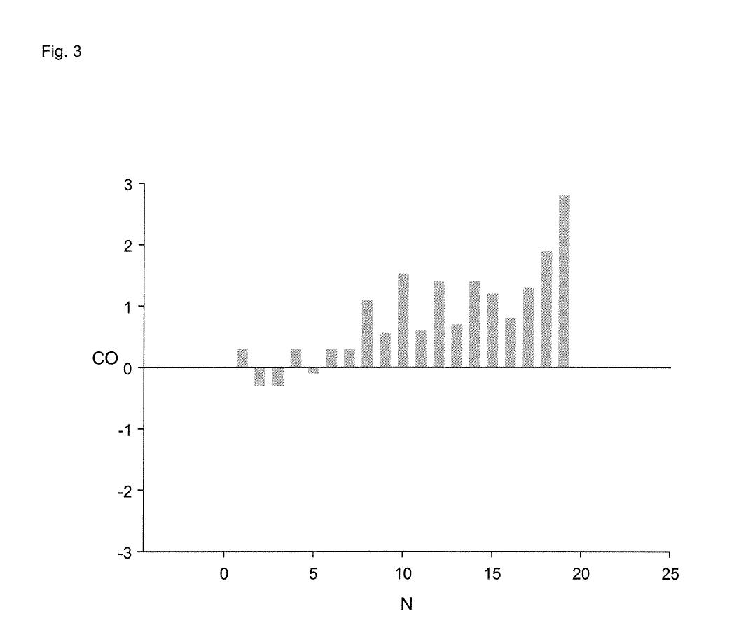

FIG. 4 depicts the change in cardiac output (CO) in patients obtaining placebo.

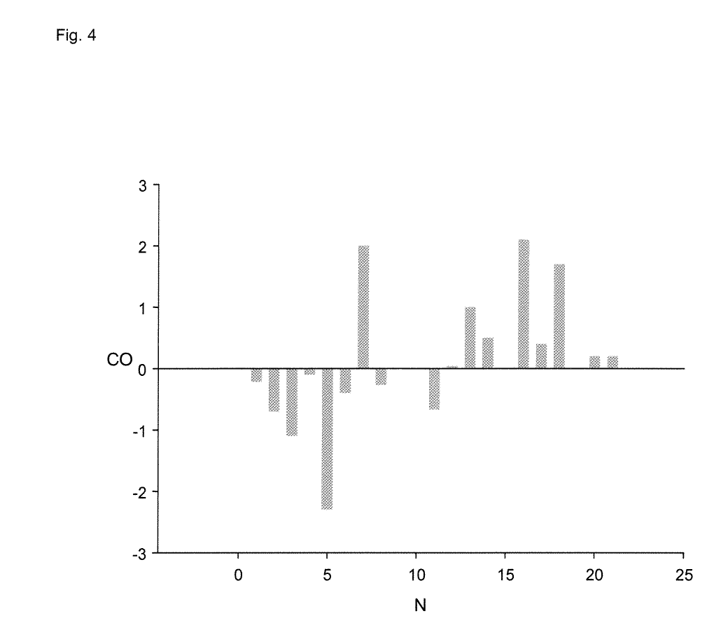

FIG. 5 depicts the change in pulmonary artery pressure (PAP) in patients obtaining Imatinib mesylate.

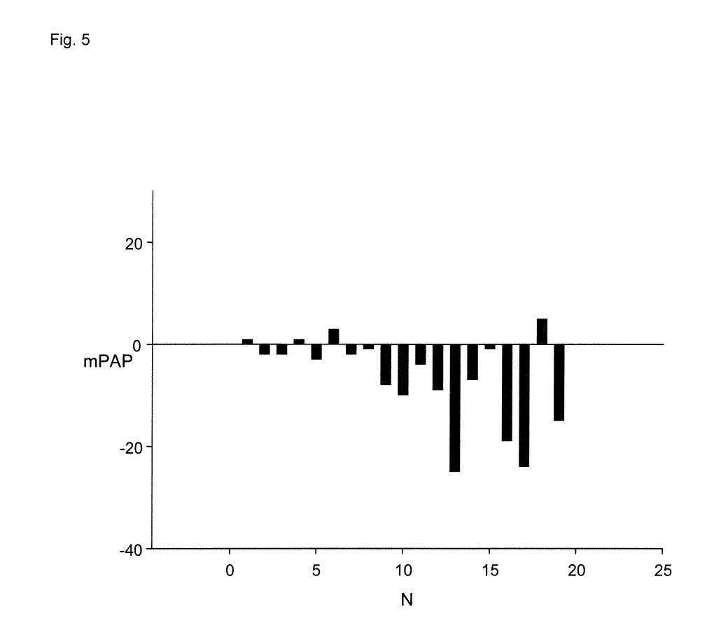

FIG. 6 depicts the change in pulmonary artery pressure (PAP) in patients obtaining placebo.

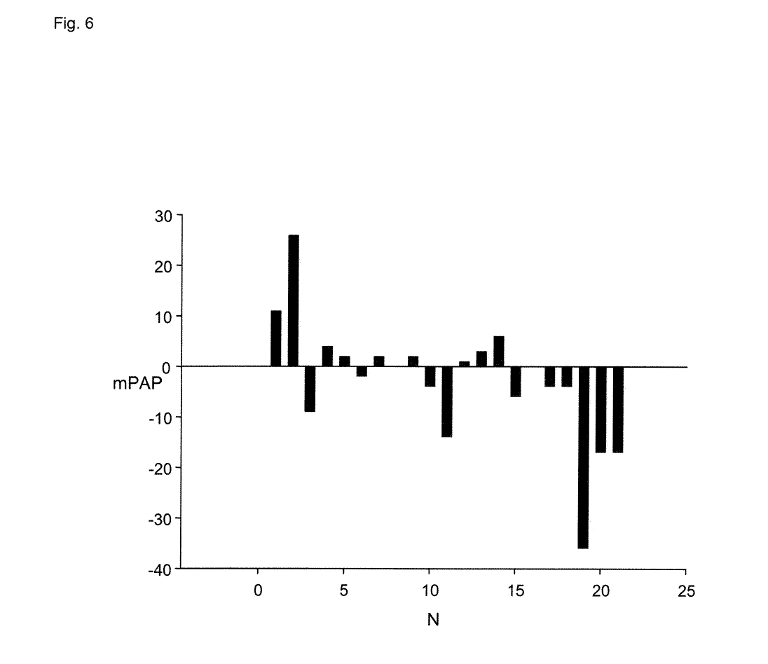

FIG. 7 depicts the patient disposition of the intention to treat (ITT) population.

FIG. 8 depicts the mean change from baseline in pulmonary hemodynamics after 6 months of treatment with imatinib or placebo. (a) mean pulmonary artery pressure (PAPm); (b) cardiac output (CO); (c) pulmonary vascular resistance (PVR); (d) 6-minute walking distance (6MWD).

FIG. 9 depicts the mean change from baseline to study end in pulmonary hemodynamics in patients randomized to imatinib or placebo, stratified by baseline PVR≧1,000 dynes·sec·cm⁻⁵ (imatinib N=8; placebo N=12) or <1,000 dynes·sec·cm⁻⁵ (imatinib N=12; placebo N=9). (a) mean pulmonary artery pressure (PAPm); (b) cardiac output (CO); (c) pulmonary vascular resistance (PVR); (d) 6-minute walking distance (6MWD).

### WORLD HEALTH ORGANIZATION CLASSIFICATION OF FUNCTIONAL STATUS OF PATIENTS WITH PULMONARY HYPERTENSION

The status of their pulmonary hypertension can be assessed in patients according to the World Health Organization (WHO) classification (modified after the New York Association Functional Classification) as detailed below:

Class I—Patients with pulmonary hypertension but without resulting limitation of physical activity. Ordinary physical activity does not cause undue dyspnea or fatigue, chest pain or near syncope.
Class II—Patients with pulmonary hypertension resulting in slight limitation of physical activity. They are comfortable at rest. Ordinary physical activity causes undue dispend or fatigue, chest pain or near syncope.
Class III—Patients with pulmonary hypertension resulting in marked limitation of physical activity. They are comfortable at rest. Less than ordinary activity causes undue dyspnea or fatigue, chest pain or near syncope.
Class IV—Patients with pulmonary hypertension with inability to carry out any physical activity without symptoms. These patients manifest signs of right heart failure. Dyspnea and/or fatigue may even be present at rest. Discomfort is increased by any physical activity.

In a preferred embodiment of the present invention the medicament is designated for treating pulmonary arterial hypertension in patients who failed prior therapy, especially after receiving at least one prostanoid, endothelin antagonist or PDE V inhibitor.

In a further preferred embodiment of the present invention the medicament is designated for treating pulmonary arterial hypertension in patients who are more severely affected, in particular in patients with Class II to Class IV functional status, more preferably Class III or IV functional status.

In a further preferred embodiment of the present invention the medicament is designated for treating pulmonary arterial hypertension in patients who are harboring BMPR2 mutations.

In a more general aspect, the present invention provides a method of treating humans suffering from
(a) idiopathic or primary pulmonary hypertension,
(b) familial hypertension,
(c) pulmonary hypertension secondary to, but not limited to, connective tissue disease, congenital heart defects (shunts), pulmonary fibrosis, portal hypertension, HIV infection, sickle cell disease, drugs and toxins (e.g., anorexigens, cocaine), chronic hypoxia, chronic pulmonary obstructive disease, sleep apnea, and schistosomiasis,
(d) pulmonary hypertension associated with significant venous or capillary involvement (pulmonary veno-occlusive disease, pulmonary capillary hemangiomatosis),
(e) secondary pulmonary hypertension that is out of proportion to the degree of left ventricular dysfunction,
(f) persistent pulmonary hypertension in newborn babies,
   especially in patients who failed prior PAH therapy, which comprises administering to said human in need of such treatment a dose effective against the respective disorder of 4-methylpiperazin-1-ylmethyl)-N-[4-methyl-3-(4-pyridin-3-yl)pyrimidin-2-ylamino)phenyl]-benzamide or a pyrimidylaminobenzamide of formula I or a pharmaceutically acceptable salt thereof, respectively, preferably a dose effective against the respective disorder of a pyrimidylaminobenzamide of formula I or a pharmaceutically acceptable salt thereof.

Depending on species, age, individual condition, mode of administration, and the clinical picture in question, effective doses, for example daily doses of about 100-1000 mg, preferably 200-600 mg, especially 400 mg of COMPOUND I, are administered to warm-blooded animals of about 70 kg bodyweight. For adult patients a starting dose corresponding to 400 mg of COMPOUND I free base daily can be recommended. For patients with an inadequate response after an assessment of response to therapy with a dose corresponding to 400 mg of COMPOUND I free base daily, dose escalation can be safely considered and patients may be treated as long as they benefit from treatment and in the absence of limiting toxicities.

The invention relates also to a method for administering to a human subject having pulmonary arterial hypertension a pharmaceutically effective amount of COMPOUND I or a pyrimidylaminobenzamide of formula I or a pharmaceutically acceptable salt thereof to the human subject. Preferably, COMPOUND I or a pyrimidylaminobenzamide of formula I or a pharmaceutically acceptable salt thereof is administered once daily for a period exceeding 3 months. The invention relates especially to such method wherein a daily dose of COMPOUND I mesylate corresponding to 100 to 1000 mg, e.g. 200 to 800 mg, especially 400-600 mg, preferably 400 mg, of COMPOUND I free base is administered.

According to the present invention, COMPOUND I is preferably in the form of the monomethanesulfonate salt, e.g. in the β-crystal form of the monomethanesulfonate salt.

The invention relates to a method of treating a warm-blooded animal, especially a human, suffering from pulmonary hypertension, especially pulmonary arterial hypertension, comprising administering to the animal a combination which comprises (a) COMPOUND I or a pyrimidylaminobenzamide of formula I and (b) at least one compound selected from compounds indicated for the treatment of pulmonary arterial hypertension, such as calcium channel antagonists, e.g. nifedipine, e.g. 120 to 240 mg/d, or diltiazem, e.g. 540 to 900 mg/d, prostacyclin, the prostacyclin analogues iloprost, flolan and treprostinil, adenosine, inhaled nitric oxide, anticoagulants, e.g. warfarin, digoxin, endothelin receptor blockers, e.g. bosentan, phosphodiesterease inhibitors, e.g. sildenafil, norepinephrine, angiotensin-converting enzyme inhibitors e.g. enalapril or diuretics; a combination comprising (a) and (b) as defined above and optionally at least one pharmaceutically acceptable carrier for simultaneous, separate or sequential use, in particular for the treatment of pulmonary arterial hypertension; a pharmaceutical composition comprising such a combination; the use of such a combination for the preparation of a medicament for the delay of progression or treatment of pulmonary arterial hypertension; and to a commercial package or product comprising such a combination.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

When the combination partners employed in the combinations as disclosed herein are applied in the form as marketed as single drugs, their dosage and mode of administration can take place in accordance with the information provided on the package insert of the respective marketed drug in order to result in the beneficial effect described herein, if not mentioned herein otherwise.

It can be shown by established test models that the COMPOUND I or a pyrimidylamino-benzamide of formula I or a pharmaceutically acceptable salt thereof, results in a more effective prevention or preferably treatment of pulmonary arterial hypertension. COMPOUND I or a pharmaceutically acceptable salt thereof has significant fewer side effects as a current therapy. Furthermore, COMPOUND I or a pharmaceutically acceptable salt thereof, results in beneficial effects in different aspects, such as, e.g. incremental benefit with time or to reverse the disease process. COMPOUND I, or a pharmaceutically acceptable salt thereof, shows an unexpected high potency to prevent or eliminate pulmonary arterial hypertension, because of its unexpected multifunctional activity, and its activity on different aspects of pulmonary arterial hypertension.

The person skilled in the pertinent art is fully enabled to select a relevant test model to prove the hereinbefore and hereinafter indicated therapeutic indications and beneficial effects (i.e. good therapeutic margin, and other advantages mentioned herein). The pharmacological activity is, for example, demonstrated by in vitro and in vivo test procedures such as rodent models of pulmonary arterial hypertension, or in a clinical study as essentially described hereinafter. The following Examples illustrate the invention described above, but are not, however, intended to limit the scope of the invention in any way.

### EXAMPLE 1

### A Randomized, Double-Blind, Placebo-Controlled Study to Evaluate the Safety and Efficacy of Six Months Treatment with the Tyrosine Kinase Inhibitor Imatinib Mesylate for the Treatment of Pulmonary Arterial Hypertension

### Primary Objectives

To assess the safety and tolerability of oral Imatinib Mesylate compared with placebo in patients with pulmonary arterial hypertension (PAH).
To evaluate efficacy of oral Imatinib Mesylate as measured by an improvement in 6-minute walk test.

### Secondary Objective(s)

To evaluate the efficacy of oral Imatinib Mesylate as measured by improvement in clinical status (assessment of WHO class and Borg Score), and changes in pulmonary homodynamic parameters (including mean pulmonary arterial pressure, mean Pulmonary Artery Wedge pressure, Systolic Arterial Pressure, Heart Rate, and Cardiac Output, Pulmonary Vascular Resistance, Systemic Vascular Resistance), time to clinical worsening, changes in plasma biomarker levels.

### Design:

In the study a total of 60 patients with PAH was enrolled who have been shown to be deteriorating on, or not tolerating, standard therapy (prostanoids (i.v., s.c., inhaled), endothelin-1 antagonists, or PDE-5 inhibitors), but may still be continuing with the standard therapy. Eligible patients were randomized to receive oral Imatinib Mesylate 200 mg daily rising to 400 mg after 2 weeks, or matching placebo. Treatment continued for 6 months with weekly visits for the first four weeks followed by monthly visits up to six months (Week 24). Safety and efficacy assessments were performed at pre-specified time points up to Week 24. Male or female patients aged 18 years or older with pulmonary arterial hypertension according to the Venice Classification (2003) of either primary (idiopathic), familial or secondary to systemic sclerosis (excluding those with marked pulmonary fibrosis) and a WHO classification of II to IV (maximum of 50% of patients will be class IV) were included. Patients harboring a mutation in BMPR2 gene were identified. Patients had been receiving therapy with prostanoids (i.v., s.c., inhaled), endothelin-1 antagonists, or PDE-5 inhibitors, but have shown to be deteriorating (not improving on), or not tolerating this standard therapy. PAH medication had been stable for at least 3 months prior to inclusion in the study (Baseline visit). Imatinib Mesylate was applied as 100 mg clinical trial formulation capsules for oral administration and matching placebo capsules. The 200 mg dose consisted of 2×100 mg capsules or 2× matching placebo. The 400 mg dose consisted of 4×100 mg capsules or matching placebo. Patients were instructed to take the study drug once daily with a meal and a large glass (8 oz/200 mL) of water and not to chew the medication, but to swallow it whole.

### Efficacy Assessments

Six minute walk test and Borg Score: Screening, Baseline, Week 4, Week 8, Week 12, Week 16, Week 20, Week 24/Study Completion.
WHO Assessment: Screening, Baseline, Week 4, Week 8, Week 12, Week 16, Week 20, Week 24/Study Completion
Hemodynamic parameters (PAP, PAWP, SAP, HR, CO, PVR and SVR) from right sided heart catheritization: Baseline and Week 24/Study Completion.

### Results

TABLE 1
Change in Key Variables Baseline to Study End (mean [percent])

| | | | PVR (dyne/s · | | |
|---|---|---|---|---|---|
| | mPAP (mmHg) | CO (l/min) | cm)−5 | PCWP (mmHg) | 6MW |
| IM | −6.42 (−11%) | 0.83 (20%) | −300 (−29%) | −0.4 (−4%) | 18.1 (5%) |
| N = 19 | | | | | |
| Placebo | −2.66 (−4%) | 0.11 (3%) | −81 (−8%) | 1.4 (19%) | −12 (−3%) |
| N = 21 | | | | | |
| IM - Placebo | −3.75 (7%) | 0.71 (17%) | 218 (−21%) | 1.8 (23%) | 30 (8%) |
| P Value | 0.27 | 0.017 | 0.029 | 0.07 | 0.06 |

TABLE 2
Change by Baseline PVR/PVR < 1000

| | mPAP | PVR | CO | 6MW |
|---|---|---|---|---|
| IM (N = 7) | −4.61538 | −173.769 | 0.291538 | 3.2 |
| PL (N = 12) | −3.25 | −74.375 | 0.57375 | 14.4 |

TABLE 3
Change by Baseline PVR/PVR > 1000

| | mPAP | PVR | CO | 6MW |
|---|---|---|---|---|
| IM (N = 12) | −8.57143 | −596.571 | 1.271429 | 70 |
| PL (N = 9) | −6.33333 | −121.75 | 0.229167 | −32 |

6MW: 6-minute walk test;
CO: cardiac output;
IM: Imatinib mesylate;;
PAP: pulmonary arterial pressure;
PCWP: pulmonary capillary wedge pressure;
PL: placebo;
PVR: pulmonary vascular resistance

The study demonstrates a clear beneficial change in pulmonary vascular resistance (PVR), cardiac output (CO) and six minute walk in response to Imatinib mesylate compared to placebo. A trend in reduction in pulmonary artery pressure (PAP) was also seen. There was a difference in the number of deaths (5 versus 3) in favor of Imatinib mesylate.

### EXAMPLE 2

### A Randomized, Double-Blind, Placebo-Controlled Trial to Evaluate Imatinib Treatment for Patients with Severe Pulmonary Arterial Hypertension with Inadequate Response to Established Therapy

### Introduction

Pulmonary arterial hypertension (PAH) (defined as a mean pulmonary artery pressure [PAPm] of ≧25 mmHg at rest or 30 mmHg with exercise, mean pulmonary capillary wedge pressure [PCWPm]≦15 mmHg and pulmonary vascular resistance [PVR]>240 dynes·sec·cm⁻⁵) leads to progressive increases in pulmonary vascular resistance (PVR), right ventricular failure and death if untreated. Estimated 1 and 3 year survival rates in idiopathic PAH (IPAH) without targeted therapy are 68% and 48%, respectively.

Current drug therapy recommendations for PAH vary depending on the patient's functional class (FC, World Health Organization's [WHO] Modification for Pulmonary Hypertension of the New York Heart Association Functional Class). The phosphodiesterase type 5 (PDE5) inhibitor sildenafil, oral endothelin receptor antagonists (ERAs) bosentan, ambrisentan and sitaxsentan, and prostacyclin analogues epoprostenol (intravenous), iloprost (inhaled) and treprostinil (subcutaneous or intravenous) are approved for patients in FC II-IV. Patients in FC III or IV who fail to improve or deteriorate with monotherapy can be treated with combination therapy, atrial septostomy and/or transplantation (lung or heart/lung). However, to date, none of these therapeutic options cure PAH despite improvement in survival; PAH remains a progressive and frequently fatal condition. Two recent meta-analyses highlighted the beneficial effects of prostacyclin analogues, ERAs and PDE5 inhibitors on exercise capacity and some other clinical endpoints in PAH patients, while only the most recent report by Galie et al. provided evidence of improved survival by the aforementioned treatments.

Pathological changes in the pulmonary arteries of patients with PAH include the formation of plexiform lesions, and smooth muscle and fibroblast proliferation leading to vascular obstruction. Platelet-derived growth factor (PDGF) is a vascular smooth muscle cell mitogen activating signal transduction pathways associated with smooth muscle hyperplasia in pulmonary hypertension. PDGF and its receptor (PDGFR) have been implicated in the pathobiology of pulmonary hypertension in animal studies and in patients with PAH thereby offering a potential new target for treatment.

Imatinib, a tyrosine kinase inhibitor that inhibits PDGFR α and β kinases, Abl, DDR and c-KIT, may therefore prove efficacious in the treatment of PAH. Several case reports have provided promising results thus warranting further study of imatinib in PAH.

In the present study the effects of imatinib versus placebo were compared in a randomized, double-blind, placebo-controlled pilot study in PAH patients who had not adequately improved with prostacyclin analogues, ERAs, PDE5 inhibitors and/or combinations of these therapies.

### Methods

### 1. Study Objectives and Design

The primary objectives were to assess the safety and tolerability of imatinib compared with placebo in PAH patients and to evaluate its efficacy using the 6-minute walk test (6MW test). Secondary objectives included changes in hemodynamic variables, and in FC.

Patients (≧18 years) in FC II-IV with idiopathic or familial PAH, or PAH associated with systemic sclerosis or congenital heart disease (WHO group I) and PVR>300 dynes·sec·cm⁻⁵ were eligible. Patients were on stable PAH medication(s) for >3 months before enrolment. Females of child-bearing potential used double-barrier contraception.

Patients with other causes of PAH were excluded. Patients were not allowed to use nonspecific PDE inhibitors, chronic inhaled nitric oxide therapy or catecholamines during the study. Additional exclusion criteria included: participation in another clinical trial within 3 months, donation or loss of blood (>400 mL) within 8 weeks or a history of another significant illness within 4 weeks. Patients were also excluded if they had pre-existing lung disease, coagulation disorders, thrombocytopenia, major bleeding or intracranial hemorrhage, history of latent bleeding risk, elevated liver transaminases (>4 times upper limit of normal [ULN]), elevated bilirubin (>2 times ULN), elevated serum creatinine (>200 μmol/L), history of elevated intracranial pressure, pregnancy, breast feeding, sickle cell anaemia, history of clinically significant drug allergy or atopic allergy, history of immunodeficiency, hepatitis B or C, or history of drug or alcohol abuse. Patients were excluded if they had known hypersensitivity to the study drug, any condition that could alter the study drug pharmacokinetics or put them at risk, if their underlying disease was likely to result in failure to survive the study, or if they were unable to perform the 6MW test due to a condition other than PAH. Eligible patients were enrolled at 7 centres in Germany, the United Kingdom, Austria, and the United States and randomized 1:1 to treatment with either imatinib or placebo.

The study was designed, implemented and reported in accordance with International Conference on Harmonization (ICH) Harmonized Tripartite Guidelines for Good Clinical Practice and all applicable local regulations (including European Directive 2001/83/EC and US Code of Federal Regulations Title 21) and with the ethical principles laid down in the Declaration of Helsinki. This study was approved by institutional review boards at all centres and all patients signed informed consent before enrolment. All deaths and safety data were reviewed throughout the study by an external data safety monitoring board.

### 2. Interventions

Treatment with imatinib (or placebo) was initiated at a dose of 200 mg orally once daily for the first two weeks of treatment. If treatment was well tolerated, the dose was increased to 400 mg/day. If the 400 mg dose was not well tolerated, down-titration to 200 mg was permitted. Patients and investigators were blind to the treatment allocation. The blinding could be broken in an emergency.

### 3. Efficacy Assessments

The primary efficacy outcome was the between-group difference in the 6MW distance (6MWD) at baseline and at 6 months. Complete hemodynamic parameters were assessed with standard techniques. FC was classified according to the WHO modification of the NYHA criteria for pulmonary hypertension.

### 4. Exploratory Analysis

To generate new hypotheses and to identify patient subgroups that may respond better than other subgroups to imatinib, additional subgroup analyses were conducted in patients with PVR values of ≧1,000 vs. <1,000 dynes·sec·cm⁻⁵ (the median of the data).

### 5. Safety Assessments

Monitoring of blood cell counts, hepatic and renal function parameters, echocardiography and cardiac magnetic resonance imaging (in selected centres) was conducted during the study. Patients were also interviewed via regular telephone calls between scheduled study visits.

### 6. Statistical Analysis

The planned sample size of 60 subjects was selected to address both safety and the primary efficacy outcome (6MWD). For the primary efficacy outcome it was estimated that the study had 80% power to detect a 55 m increase in the 6MWD with 95% confidence (two-sided p<0.05), based on a standard deviation (SD) of 75 m.

Analyses were conducted within the intention-to-treat (ITT) population, which consisted of all patients who received at least one dose of study medication. Dropouts were excluded from the analysis The primary efficacy analysis (6MWD) was performed using analysis of covariance (ANCOVA) with baseline value as a covariate. ANCOVAs were also used to assess between-group differences in pulmonary hemodynamics and blood gases. Missing data were not imputed so only subjects with assessment both at baseline and post-treatment were included in the ANCOVA analysis. FC was compared using Fisher's test.

In addition, exploratory analyses (post-hoc) were performed in subgroups classified according to baseline PVR values ≧ or <1,000 dynes·sec·cm⁻⁵ at baseline (i.e. the median PVR in the study).

### Results

### 1. Disposition and Baseline Characteristics:

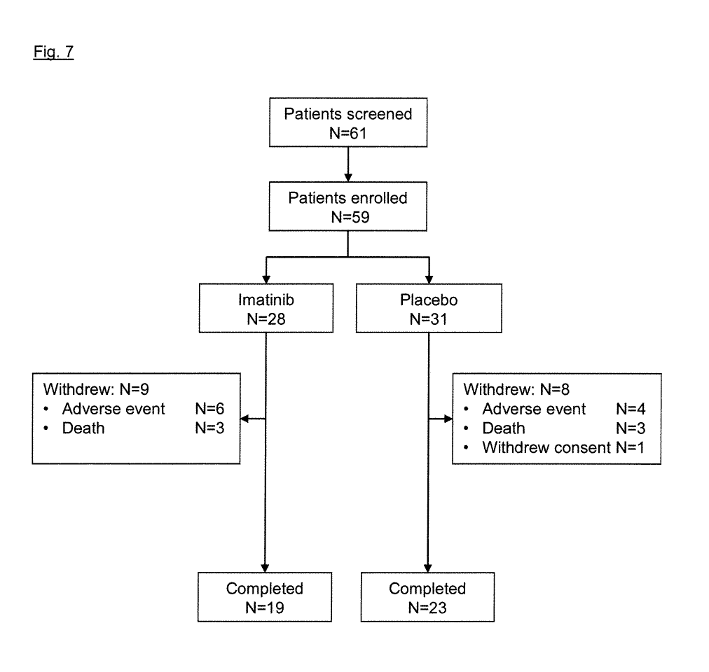

Fifty-nine patients (40 female; 19 male) were enrolled with 42 (71.2%) completing the 6 month study (FIG. 7). The majority of dropouts not related to death were to worsening of PAH. Baseline characteristics were similar between the two treatment groups (Table 4). Overall, patients had a mean age of 44.3 years, mean weight of 68.7 kg and mean body mass index of 24.6 kg/m². Fifty five of the 59 patients were Caucasian and 78% had idiopathic PAH (Table 4). At baseline, 79% of the imatinib- and 81% of the placebo-group patients were receiving combination therapy (Table 4).

TABLE 4
Baseline characteristics of the intention to treat (ITT) population

| | Imatinib | Placebo |
|---|---|---|
| | (N = 28) | (N = 31) |
| Age (years), mean (SD) | 44.4 (15.3) | 44.2 (15.7) |
| Gender, male/female, n (%) | 10 (36)/8 (64) | 9 (29)/22 (71) |
| Ethnicity, n (%) | | |
| Caucasian | 26 (92) | 29 (94) |
| Asian | 0 | 1 (3) |
| Black | 1 (4) | 0 |
| Pacific Islander | 0 | 1 (3) |
| Hispanic | 1 (4) | 0 |
| Weight (kg), mean (SD) | 70.1 (14.7) | 67.4 (23.4) |
| Height (cm), mean (SD) | 168.6 (8.8) | 164.3 (8.6) |
| Diagnosis, n (%) | | |
| Idiopathic pulmonary hypertension | 21 (75) | 25 (81) |
| Familial pulmonary hypertension | 2 (7) | 0 |
| Pulmonary hypertension secondary | 1 (4) | 5 (16) |
| to systemic sclerosis | | |
| Other | 4 (14) | 1 (3) |
| WHO classification, n (%)* | | |
| Class II | 13 (48) | 7 (23) |
| Class III | 12 (44) | 23 (74) |
| Class IV | 2 (7) | 1 (3) |
| PAH specific treatments, n (%) | | |
| ERA alone | 2 (7) | 4 (13) |
| Sildenafil alone | 2 (7) | 0 (0) |
| Prostacylin analog alone | 2 (7) | 1 (3) |
| ERA + prostacylin analog | 1 (4) | 3 (10) |
| ERA + sildenafil | 12 (43) | 9 (29) |
| Sildenafil + prostacyclic analog | 5 (18) | 3 (10) |
| ERA + sildenafil + prostacyclin | 4 (14) | 10 (32) |
| Calcium channel blocker | 0 | 1 (3) |

SD: standard deviation;
PH: pulmonary hypertension;
prostacyclin analogues (iloprost, epoprostenol, trepostinil and beraprost);
ERA: endothelin receptor antagonists (bosentan and ambrisentan)
*WHO assessment was not available for one patient receiving imatinib

### 2. Efficacy Outcomes:

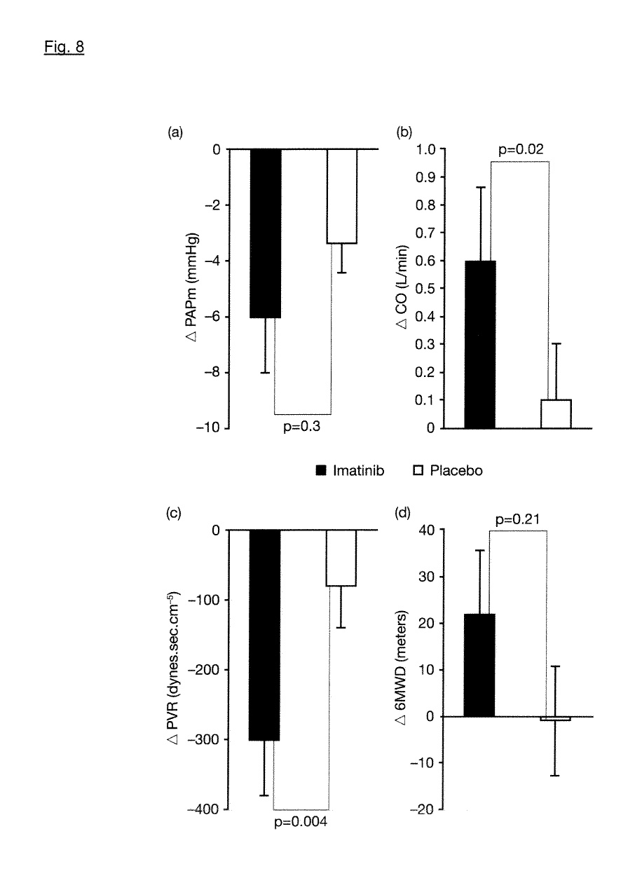

The mean (±SD) 6MWD did not significantly change in the imatinib group vs. placebo (+22±63 vs. −1.0±53 m; mean treatment difference 21.7 m; 95% Cl (−13.0, 56.5); p=0.21) (Table 5; FIG. 8). There was, however, a significant decrease in PVR (mean treatment difference −230.7 dynes; 95% Cl (−383.7, −77.8; p=0.004) and increase in cardiac output (CO; mean treatment difference 0.68 L/min; 95% CI (0.10, 1.26; p=0.02) in imatinib recipients compared with placebo (FIG. 8). There was no significant difference in PAPm (FIG. 8) or change in FC between imatinib and placebo treated patients (data not shown).

There was an increase in arterial and mixed venous oxygen saturation (p<0.05) with imatinib. Systemic arterial oxygen saturation increased from 88±9% to 93±5% with imatinib treatment compared with no change with placebo (92±4% at baseline vs. 92±3% at end of study) (mean treatment difference 2.4%; 95% CI (0.5, 4.3)); mixed venous oxygen saturation increased from 58±10% to 65±7% with imatinib treatment (consistent with the increase in CO) compared with a decrease with placebo (61±6% at baseline vs. 57±9% at end of study) (mean treatment difference 7.0%; 95% CI (2.1, 11.9)).

TABLE 5
Six-minute walking distance (6MWD) observed at baseline and end of study, and
changes from baseline following imatinib and placebo therapy in patients with
PAH. The change is expressed as the average alteration in 6MWD from baseline.

| | Imatinib | | Placebo | | | |
|---|---|---|---|---|---|---|
| | Distance | Change vs. | Distance | Change vs. | Treatment | |
| | walked (m), | baseline (m) ^{a} | walked (m), | baseline (m) ^{a} | difference | |
| | mean (SD) | mean (SD) | mean (SD) | mean (SD) | (m) ^{b} | p-value ^{b} |
| Baseline | 392 (89) | — | 369 (118) | — | — | — |
| | N = 28 | | N = 29 | | | |
| Study end | 419 (85) | 22 (63) | 399 (86) | −1 (53) | 21.7 | 0.21 |
| | N = 21 | N = 21 | N = 22 | N = 21 | | |

^{a} Patients with both a baseline and end of study assessment.
^{b} ANCOVA of ITT population

### 3. Exploratory Subgroup Analyses:

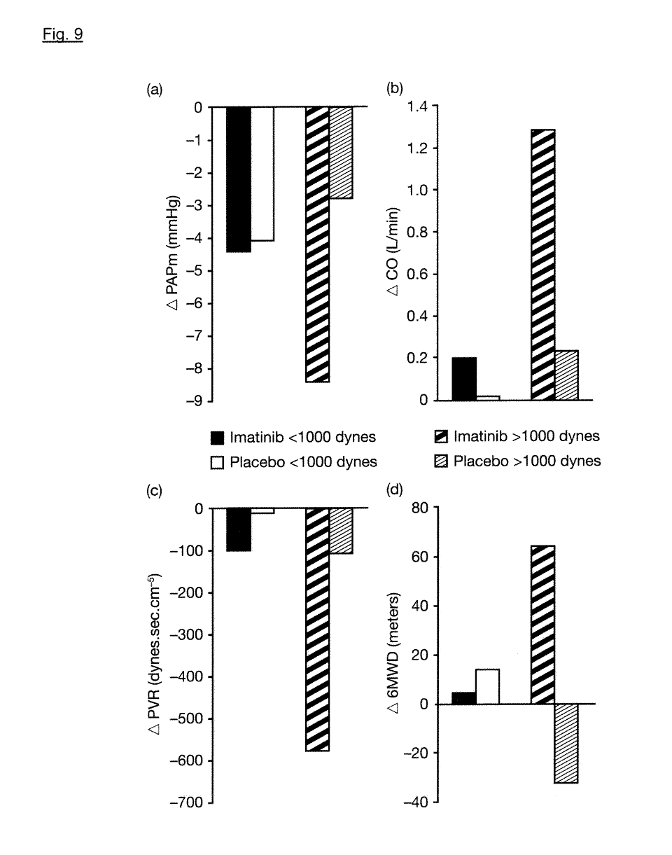

In patients with a baseline PVR≧1,000 dynes·sec·cm⁻⁵, there was a substantial improvement between baseline and study end for PAPm, CO, PVR and 6MWD in the imatinib group compared with placebo (FIG. 9). However, among patients with a baseline PVR<1,000 dynes·sec·cm⁻⁵, no major differences between baseline and study end for PAPm, CO, PVR or 6MWD were observed (FIG. 9).

### 4. Safety and Tolerability:

The most common adverse events (AEs) observed in this clinical study were as expected for this population and this drug. The most common AEs reported in the imatinib group were nausea (N=14; 50%), headache (N=10; 35.7%) and peripheral edema (N=7; 25.0%). These AEs did not lead to discontinuation of study drug. Nausea was controlled by taking the medication with food. A total of 21 (75%) patients in the imatinib group and 24 (77%) patients in the placebo group reported AEs of mild intensity, 20 (71%) in the imatinib group and 19 (61%) in the placebo group patients reported AEs of moderate intensity, and 9 (32%) patients in the imatinib group and 5 (16%) patients in the placebo group reported AEs of severe intensity. Serious AEs (SAEs) were reported for 11 imatinib recipients (39%) and 7 placebo recipients (23%). SAEs in the imatinib group included cardiac arrest (N=2), vertigo (n=1), pancreatitis (N=1), catheter related complication (N=1), liver dysfunction (N=2), dizziness (N=1), presyncope (N=1), syncope (N=1), haemoptysis (N=1), worsening pulmonary hypertension (N=3), and arterial rupture (N=1). SAEs in the placebo group included atrial flutter (N=1), cardiac arrest (N=2), right ventricular failure (N=2), general physical health deterioration (N=1), fluid retention (N=1), dizziness (N=1), and worsening pulmonary hypertension (N=3).

Overall there was a fall in the haemoglobin levels with imatinib (151±14 to 128±16 g/L, SD) and a rise in hemoglobin levels with placebo (143±25 to 152±25 g/L). There were no relevant changes over time on the following variables: white blood cell count, platelet count albumin, alkaline phosphatase, total bilirubin, calcium, cholesterol, creatinine, g-GT, glucose, lactate dehydrogenase, inorganic phosphorus, lipase, amylase, potassium, total protein, C-reactive protein, glutamate oxalacetate transaminase, glutamate pyruvate transaminase, sodium, triglycerides, urea, and uric acid.

There were three deaths in each group. Two additional patients died in the placebo group within 2 months of completing the study. One patient in the imatinib group and one patient in the placebo group had rupture of the pulmonary artery (fatal in both cases).

### Discussion

This is the first randomized, double-blind, placebo controlled trial to assess the safety, tolerability and efficacy of the tyrosine kinase inhibitor imatinib in patients with PAH. Although imatinib appeared safe and well tolerated over a 6 month period, the primary efficacy parameter (6MWD) did not improve in patients randomized to imatinib compared with placebo, despite significant improvement in secondary endpoints.

### Treatment Efficacy

Overall, 59 patients were enrolled. As per study protocol, only patients on background treatment with at least one PAH specific drug (i.e. prostacyclin analogues, ERAs, PDE5 inhibitors) who had not adequately improved were enrolled (56% of patients were receiving two drugs and 24% receiving three drugs at baseline). This may have contributed to the reduced improvement in 6MWD observed in this study compared with previous studies in which only treatment naïve patients were included. In clinical trials in which background specific medications have been allowed, the overall improvement in 6MWD has been less than in the treatment naïve trials.

### Safety Aspects

It has been suggested that inhibition of the ABL tyrosine kinase pathway may infrequently induce myocardial damage in patients receiving long-term treatment with imatinib for chronic myelogenous leukemia (CML). However, a long-term, multicenter study in a large population of patients with CML showed an acceptable safety profile for imatinib. A review of all patients receiving imatinib shows that 0.5% of patients per year developed incident congestive cardiac failure (no risk factors present). In patients with CML receiving imatinib, 0.4% of patients per year develop congestive cardiac failure compared with 0.75% per year for patients receiving interferon gamma plus Ara-C. Considering the potential for cardiotoxicity which could be even more problematic for patients with PAH, regular assessments of cardiac function by echocardiography and measurements of serum cardiac troponin levels were performed in this trial. Overall, there were no signals indicating a potential detrimental effect of imatinib on myocardial function when compared to the overall safety profile of the placebo group. In contrast, some of the beneficial effect of imatinib on PVR reduction appeared to be due to improvements in CO, suggestive of improved right ventricular contractility in patients with PAH. Nonetheless, cardiac safety remains a key concern with other kinase inhibitors, such as sunitinib.

### Exploratory Subgroup Analysis

Although no significant increases in 6MWD were observed with imatinib compared with placebo, significant improvements in CO and PVR were observed. These observations led us to undertake a post-hoc analysis stratifying patients by baseline PVR. In patients with baseline PVR≧1,000 dynes·sec·cm⁻⁵, there was a substantial improvement from baseline to study end for 6MWD, PVR, and CO in the imatinib group, when compared with placebo (FIG. 9). This was not observed in the patients with PVR levels <1,000 dynes·sec·cm⁻⁵.

However, these results have to be interpreted with caution as this was an unplanned analysis. In addition, tyrosine kinase inhibitors are not recognized to have any significant vasodilator or inotropic effects, with their effects considered anti-proliferative and pro-apoptotic. One hypothesis that could explain the current study results is that for treatment with imatinib to be effective, a certain degree of disease severity (i.e. vascular proliferation) may be needed. However, as these data are hypothesis generating, it cannot be excluded that less severe patients with PAH may also benefit from long-term imatinib therapy via a preventive mechanism.

### Conclusion and Perspective

The results of this pilot study suggest that imatinib is safe and well tolerated in patients with PAH. In addition, the efficacy analyses provide proof of concept supporting the use of agents targeting proliferative growth factor pathways in PAH.

1-11. (canceled)

12. A method of treating humans suffering from pulmonary arterial hypertension (PAH) in patients who failed prior PAH therapy, which comprises administering to a said human in need of such treatment a dose effective against PAH of a pyrimidylaminobenzamide of formula I 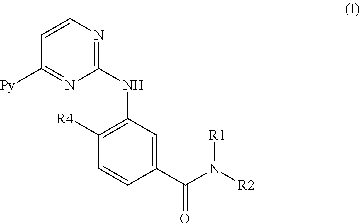
wherein
Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
or a pharmaceutically acceptable salt thereof.

13. The method according to claim 12, wherein the pyrimidylaminobenzamide of formula I is 4-methyl-3-[[4-(3-pyridinyl)-2-pyrimidinyl]amino]-N-[5-(4-methyl-1H-imidazol-1-yl)-3-(trifluoromethyl)phenyl]benzamide.

14. The method according to claim 12, wherein the pyrimidylaminobenzamide of formula I is 4-methyl-3-[[4-(3-pyridinyl)-2-pyrimidinyl]amino]l-N-[5-(4-methyl-1H-imidazol-1-yl)-3-(trifluoromethyl)phenyl]benzamide in the form of its hydrochloride monohydrate.

15. The method according to claim 12, wherein prior PAH therapy included receiving at least one prostanoid, endothelin antagonist or PDE V inhibitor.

16. The method according to claim 12, wherein the pyrimidylaminobenzamide of formula I is designated for treating PAH in patients who are more severely affected.

17. The method according to claim 12, wherein the pyrimidylaminobenzamide of formula I is designated for treating PAH in patients who are harboring BMPR2 mutations.

18. Use of a pyrimidylaminobenzamide of formula I 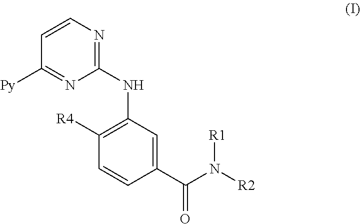
wherein
Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
or a pharmaceutically acceptable salt thereof, for the manufacture of a medicament for treating pulmonary arterial hypertension (PAH) in patients who failed prior PAH therapy.
